(19) United States
(12) Patent Application Publication
(10) Pub. No.: US 2026/0063649 A1
(43) Pub. Date: Mar. 5, 2026

(54) PROCESS FOR DETERMINING PREGNANCY AND/OR A PREGNANCY STAGE OF AN UNGULATE FROM ITS FECES

(71) Applicants: Université de Liège, Liège (BE); Centre Hospitalier Universitaire de Liège, Liège (BE)

(72) Inventors: Jérôme PONTHIER, Embourg (BE); Etienne CAVALIER, Hermée (BE); Vincent FRISÉE, Ouffet (BE); Patrice DUFOUR, Hermée (BE); Caroline LE GOFF, Liège (BE)

(21) Appl. No.: 18/819,075

(22) Filed: Aug. 29, 2024

(51) Int. Cl. G01N 33/74

(52) U.S. Cl. CPC G01N 33/743; G01N 2800/36

(57) ABSTRACT

The present invention relates to a process for determining pregnancy and/or a pregnancy stage of an ungulate from its feces.

### FIELD OF THE INVENTION

The present invention relates to a process for determining pregnancy and/or a pregnancy stage of an ungulate from its feces.

### BACKGROUND OF THE INVENTION

For ungulates, easy and non-invasive pregnancy diagnosis/determination technique is required to sort pregnant and non-pregnant females before culling or to assess fertility, notably in wild groups. However, ungulates are often extensively bred and simple manipulations like blood sampling or Trans-Rectal Palpation and Ultrasonography (TRPUS) are time-consuming, dangerous for animals and operators, because of ungulates comportment, thus requiring heavy contention methods associated with important workforce.

In the case of American Bison, due to its wild character and its extensive breeding management, blood samples availability is limited. In a natural environment, American Bison calving season is spring (May to July) after around 260 to 285 days of gestation (Kirkpatrick, J. F., et al., 1992. Pregnancy and ovulation detection in bison (Bison bison) assessed by means of urinary and fecal steroids. J Wildl Dis 28, 590-597), although breeding season, grazing areas and nutrition could modulate this duration.

The endocrinology of American Bison and bovine gestations seems to share common features, such as production of Pregnancy Associated Glycoproteins (PAG) (Frisee, V., et al., 2022. American Bison (Bison bison) reproductive endocrinology: serum Pregnancy Associated Glycoproteins (PAG), Progesterone, Estrone and Estrone-Sulfate in non pregnant animals and during gestation. Domest Anim Endocrinol 78, 106684) and estrogens (Dufour, P., et al., 2021. Development and validation of a liquid chromatography coupled to mass spectrometer (LC-MS) method for the simultaneous quantification of estrone-3-sulfate, progesterone, estrone and estradiol in serum of mares and American bisons. Res Vet Sci 136, 343-350; Frisee, V., et al., 2022. American Bison (Bison bison) reproductive endocrinology: serum Pregnancy Associated Glycoproteins (PAG), Progesterone, Estrone and Estrone-Sulfate in non pregnant animals and during gestation. Domest Anim Endocrinol 78, 106684; Kirkpatrick, J. F., et al., 1992. Pregnancy and ovulation detection in bison (Bison bison) assessed by means of urinary and fecal steroids. J Wildl Dis 28, 590-597).

Estrogens are a complex class of steroid hormones, characterized by an aromatic A ring and an alcohol group on the 3-carbon. Mammals use 5 alpha-aromatase to transform androgens in estrogens: e.g. estrone (E1) is directly derived from androstenedione. In numerous mammals' species, the placenta produces different estrogens that can be conjugated with sulfate or glucuronate chains and that can be assayed for pregnancy diagnosis. Estrone (E1) and estrone-sulfate (E1S) productions were observed in 2^{nd} and 3^{rd} trimesters of pregnancy, but E1 seemed to be a residue of the placental production of E1S. Despite E1S interindividual variability, it was constantly increasing in American Bison sera during pregnancy (Frisee, V., et al., 2022. American Bison (Bison bison) reproductive endocrinology: serum Pregnancy Associated Glycoproteins (PAG), Progesterone, Estrone and Estrone-Sulfate in non pregnant animals and during gestation. Domest Anim Endocrinol 78, 106684).

As stated above, collection of blood samples requires gathering and immobilization that could induce stress and injuries in ungulates, in particular in

American Bison. Urinary and fecal sexual steroids assays are potential alternative solutions already tested on small numbers of American Bison at the third month of pregnancy. In practice, urine is difficult to collect and only total estrogens were assayed in pregnant Bison cows' feces, without information about the type of estrogen produced (Kirkpatrick, J. F., et al., 1992. Pregnancy and ovulation detection in bison (Bison bison) assessed by means of urinary and fecal steroids. J Wildl Dis 28, 590-597). Immuno-assays were used (Kirkpatrick, J. F., et al., 1992. Pregnancy and ovulation detection in bison (Bison bison) assessed by means of urinary and fecal steroids. J Wildl Dis 28, 590-597), and, they are not always properly validated to determine steroids concentration in the species' biological matrix (Dufour, P., et al., 2021. Development and validation of a liquid chromatography coupled to mass spectrometer (LC-MS) method for the simultaneous quantification of estrone-3-sulfate, progesterone, estrone and estradiol in serum of mares and American bisons. Res Vet Sci 136, 343-350), which may cause variations in the observed levels.

Consequently, nowadays, there is a real need for an easy and non-invasive pregnancy diagnosis technique allowing to avoid gathering and immobilization for difficult samplings, while ensuring a high specificity, a high sensitivity and a high precision.

### SUMMARY OF THE INVENTION

It is an object of the invention to overcome at least partly the abovementioned drawbacks and to provide a reliable non-invasive pregnancy diagnosis technique for ungulates.

More particularly, it is an object of the invention to provide a process for determining pregnancy and/or a pregnancy stage of an ungulate without gathering and immobilization for difficult samplings, while ensuring a high specificity, a high sensitivity and a high precision.

To this end, according to the invention, there is provided a process for determining pregnancy and/or a pregnancy stage of an ungulate from its feces, comprising:
an extraction of steroids from an ungulate's feces sample previously taken or collected to obtain an extract comprising steroids,
a dosage of estrone-sulfate steroid (E1S) present in the extract by a separative method coupled to mass spectrometry to determine an estrone-sulfate steroid (E1S) concentration in the extract,
a determination of the pregnancy and/or of the pregnancy stage of the ungulate based on the estrone-sulfate steroid (E1S) concentration with reference to a predetermined threshold value estrone-sulfate steroid (E1S) concentration above which the ungulate is determined as being pregnant.

By the terms “pregnancy stage”, it is meant in the sense of the present invention the trimester or the month of pregnancy/gestation reached.

The threshold value estrone-sulfate steroid (E1S) concentration above which the ungulate is determined as being pregnant is determined by a comparative analysis between a group of pregnant ungulate samples and a group of non-pregnant ungulate samples for which a significant difference in estrone-sulfate steroid (E1S) concentration is observed/measured.

According to the present invention, the dosage of estrone-sulfate steroid (E1S) present in the extract by a separative method coupled to mass spectrometry to determine an estrone-sulfate steroid (E1S) concentration in the extract, may be a quantitative or a semi-quantitative dosage.

The process according to the present invention is a non-invasive process, allowing to avoid gathering and immobilization for difficult samplings since ungulate's feces sample are mainly taken or collected on the ground. Also, contrary to current practices, a process according to the present invention ensures a high specificity, a high sensitivity and a high precision because at least one specific steroid, in this case estrone-sulfate (E1S), is targeted allowing to avoid cross-reactions with other steroids as this is the case when dosing total estrogens.

The invention is defined by the independent claims. The dependent claims define advantageous embodiments.

Preferably, according to the invention, the ungulate's feces sample is stored at a temperature inferior to 15° C., preferably at a temperature inferior to 10° C., within 6 hours of taking or collecting the sample before the extraction of steroids from the feces sample. It has been shown, in the context of the present invention, that such a storage avoids modification of steroids (conjugated or not) concentrations in feces, observed when stored at room temperature.

More preferably, according to the invention, the ungulate's feces sample is frozen at a temperature inferior to 0° C., preferably at a temperature inferior to −10° C., more preferably at a temperature ranging from −20° C. to −80° C., even more preferably at a temperature of −80° C., within 6 hours of taking or collecting the sample before the extraction of steroids from the feces sample. It has been shown, in the context of the present invention, that such a frozen step avoids modification of steroids (conjugated or not) concentrations in feces, observed when stored at room temperature.

Advantageously, according to the invention, the extraction of steroids from the ungulate's feces sample is performed with an organic solvent, for example with methanol.

Preferably, according to the invention, the extract comprising steroids is washed with a washing solution, for example with hexane.

Advantageously, according to the invention, the separative method is a chromatographic method, for example gas chromatography or liquid chromatography or high performance liquid chromatography or thin layer chromatography.

Preferably, according to the invention, the predetermined threshold value estrone-sulfate steroid (E1S) concentration ranges from 10 pg/g to 500 pg/g. More preferably, the predetermined threshold value estrone-sulfate steroid (E1S) concentration is set at 500 pg/g, preferably at 450 pg/g, preferably at 400 pg/g, preferably at 350 pg/g, preferably at 300 pg/g, preferably at 250 pg/g, preferably at 200 pg/g, preferably at 150 pg/g, preferably at 100 pg/g, preferably at 90 pg/g, preferably at 80 pg/g, preferably at 70 pg/g, preferably at 60 pg/g, preferably at 50 pg/g, preferably at 40 pg/g, preferably at 30 pg/g, preferably at 20 pg/g, preferably at 10 pg/g,

According to the invention, a tolerance of 25%, preferably a tolerance of 20%, more preferably a tolerance of 15%, even more preferably a tolerance of 10% is applied (is accepted) for the predetermined threshold value estrone-sulfate steroid (E1S) concentration.

Advantageously, according to the invention, testosterone as an additional steroid is dosed simultaneously to the dosage of estrone-sulfate steroid (E1S) in the extract by the separative method coupled to mass spectrometry to determine the concentration in the extract of said testosterone as an additional steroid. Such a dosage of testosterone allows to determine if the ungulate's feces sample previously taken or collected is from a male or a female.

Preferably, according to the invention, the ungulate is the American Bison (Bison bison bison or Bison bison athabascae).

The present invention also relates to a kit for determining pregnancy and/or a pregnancy stage of an ungulate from its feces, comprising:
an extraction buffer for extracting steroids from an ungulate's feces sample,
a reconstitution buffer for reconstituting the extracted steroids in a precise volume of solution compatible with a separative method coupled to mass spectrometry,
a separation means, in particular a chromatographic column, for separating the steroids with the separative method coupled to mass spectrometry,
a least one mobile phase for transporting the extracted steroids through the separation means, in particular through the chromatographic column,
an internal standard mix being a mixture of isotopically labelled compounds, chemically similar to the steroids of interest, said internal standard mix comprising at least estrone-sulfate steroid (E1S) isotopically labelled,
a calibrator solution for calibrating the separative method coupled to mass spectrometry.

Preferably, a kit according to the present invention further comprises a precolumn.

Advantageously, a kit according to the present invention further comprises at least one control solution for monitoring the accuracy and precision of the separative method coupled to mass spectrometry.

Preferably, the internal standard mix of a kit according to the present invention further comprises testosterone isotopically labelled.

Advantageously, a kit according to the present invention further comprises a rinsing solution for rinsing the separation means and the system used for the separative method coupled to mass spectrometry. Typically, such a rinsing solution is used to clean the separation means (for example the chromatographic column) and to clean the system used for the separative method coupled to mass spectrometry (for example the LC-MS/MS system) between analyses to avoid cross-contamination between samples.

Preferably, a kit according to the present invention further comprises a well plate, for example a multi-well plate for containing the samples ready for analysis with the separative method coupled to mass spectrometry. Such a plate is placed in an autosampler which injects the samples into the separation means, in particular in the chromatographic column.

Typically, the internal standard mix is added to the samples before analysis with separative method coupled to mass spectrometry. The isotopically labelled compounds, chemically similar to the steroids of interest, allow to correct analytical variations and to ensure accurate quantification of steroids.

Typically, the reconstitution buffer is used after evaporation of the extraction buffer to concentrate the samples.

According to the invention, the kit may comprise several control solutions, preferably a level 1 control solution (low concentrations solution), a level 2 control solution (medium concentrations solution) and a level 3 control solution (high concentrations solution) for monitoring the accuracy and precision of the separative method coupled to mass spectrometry, notably in clinical diagnostics, for the quantitative determination of steroids in the sample.

Preferably, according to the invention, the calibrator solution comprises at least one level of concentration, preferably several levels of concentrations, and a blank. Such a calibrator solution is designed to calibrate the measurement method, notably in clinical diagnostics, for the quantitative determination of steroids in the sample.

### SHORT DESCRIPTION OF THE DRAWINGS

These and further aspects of the invention will be explained in greater details by way of examples and with reference to the accompanying figures in which:

FIG. 1 shows the correlation between estrone-sulfate (E1S) concentration measured in serum and the one determined in feces samples.

FIG. 2 shows the estrone-sulfate (E1S) concentration evolution in feces (in pg/g) assayed by Liquid Chromatography coupled to Mass Spectrometry during American Bison pregnancy in days. Each dot represents the E1S concentration observed in a different pregnant American Bison cow for its day of pregnancy at sampling.

### DETAILED DESCRIPTION OF EMBODIMENTS OF THE INVENTION AND EXAMPLES

### 1. Material and Methods

### Animals and Sampling

Animals are freely grazing in meadows previously dedicated to beef cattle. The first breeding herd is divided in 2 separate meadows of 19 and 21 ha, each welcoming 2 sexually mature American Bison bulls and respectively 25 and 26 sexually mature cows. In the second ranch, 10 sexually mature American Bison cows are roaming freely with one sexually mature bull in a 15 ha meadow. American Bison are not used to human presence and to manipulations. Once a year, animals are gathered and immobilized for mandatory diseases screening in a specific handling/chute system. Between January and February 2019, and between February and March 2020, this opportunity was used to collect blood in 2 dry tubes by venipuncture under the tail of all matures American Bison. Only mature animals which reached sufficient size to be pubescent were sampled. Blood samples were centrifuged (1000×g for 10 min) and stored frozen (−80° C.) until assays. Feces were also collected by rectal palpation (±50 mL) and were stored frozen (−20° C.) within 6 hours after collection until assays. Among the 5 American Bison bulls, 2 were sampled in 2019 and 2020. Among the 93 serum samples of American Bison cows, 52 were collected twice on the same animal in 2019 and 2020. Of the 73 feces samples of American Bison cows, 10 were collected twice on the same animal in 2019 and 2020.

Calving days of the American Bison cows were recorded for the year following the sampling procedures. Fertilization day of cows that calved was retrospectively obtained by subtracting 276 days of the calving day. Gestation stage/pregnancy stage on sampling day was then determined by subtracting the fertilization day of the sampling day. Other events, such as abortion, disease, culling or prolonged gestation (more than 276 days after sampling) were reported by the breeders during the following year.

### Chemicals and Reagents

Reference standard and internal standard solutions were prepared from powder of E1S sodium salt and d4-estrone-3-sulfate sodium salt (Sigma-Aldrich, St. Louis, MO, USA) dissolved in methanol. Water, acetonitrile, hexane and methanol (LC-MS grade) were purchased from Biosolve (Biosolve, Dieuze, France). Ammonia solution (25% in water) (Merck KGaA, Darmstadt, Germany) was Suprapur® grade and was purchased from Sigma-Aldrich (St. Louis, MO, USA). Controls and calibrator samples were made by spiking standards in 500 mg of feces collected from male cattle (Bos taurus) before extraction.

### Estrone-Sulfate (E1S) Assays in Serum

Sexual steroid E1S was assayed with the validated and described for American Bison LC-MS technique (Dufour, P., et al., 2021. Development and validation of a liquid chromatography coupled to mass spectrometer (LC-MS) method for the simultaneous quantification of estrone-3-sulfate, progesterone, estrone and estradiol in serum of mares and American bisons. Res Vet Sci 136, 343-350). Lower Limit of Quantification (LLOQ), defined as the lowest concentration in the validation standards that reported Relative Standard Deviation (RSD) and Relative Bias (RB) lower than 15%, is established at 0.1 ng/ml (Dufour, P., et al., 2021. Development and validation of a liquid chromatography coupled to mass spectrometer (LC-MS) method for the simultaneous quantification of estrone-3-sulfate, progesterone, estrone and estradiol in serum of mares and American bisons. Res Vet Sci 136, 343-350; Frisee, V., et al., 2022. American Bison (Bison bison) reproductive endocrinology: serum Pregnancy Associated Glycoproteins (PAG), Progesterone, Estrone and Estrone-Sulfate in non pregnant animals and during gestation. Domest Anim Endocrinol 78, 106684).

### Feces Sample Preparation and LC-MS

500 mg (±50 mg, weighed precisely) of calibrator, quality control or American Bison's feces sample were spiked with 10 μL of internal standard mixture (d4-E1S at 10 ng/ml) before extraction. 2 mL of methanol were added to the spiked feces, the mixture was vortexed for 10 sec and then sonicated for 15 min. Mixture was centrifuged (1000×g during 5 min) and the supernatant was collected and transferred to a glass tube. The extraction by methanol was repeated once and the methanol fractions were gathered. The methanol extract was washed with 3 mL of hexane and then evaporated to dryness under vacuum at 40° C. overnight. The dry extract was then resolubilized in 100 μL of a water/acetonitrile (80/20) mixture and transferred in a 2 mL Eppendorf. The mixture was centrifuged at 16100×g during 10 min and the supernatant was transferred to LC vials before injection of 40 μL of the mixture into the LC-MS device.

The chromatographical separation took place on a Shimadzu Nexera X2 LC-30 CE (Shimadzu Co., Kyoto, Japan) equipped with a BEH C18 column (2.1 mm×100 mm, 1.7 μm particle size) (Acquity UPLC, Waters). Mobile phase A was 0.02% NH₄OH in water and mobile phase B was acetonitrile. The flow rate was 0.4 mL/min and the separation was achieved by using a gradient mode. The initial mobile phase composition was 20% of B and was held for 0.1 min. Between 0.1 min and 7 min the percentage of B linearly grew to 95%, proportion held for 3 min. Between each sample, column was allowed to equilibrate with the initial condition of gradient for 1 min. The HPLC system was connected to a linear combination of triple quadrupole and OrbiTrap mass analyzer, QTrap 6500 (ABSciex, Framingham, Massachusetts, USA) operating in triple-quadrupole mode. The resolution of quadrupoles Q1 and Q3 was set to unit. An ESI source operating in negative ionization mode during 3 min and then positive mode during 6 min was employed, the ion spray voltage was −4500 V (negative mode) or 5500 V (positive mode), gases 1, 2 and curtain gas were 30, 70 and 30, respectively and the ion source temperature was 650° C. The LC-MS conditions are gathered in Table 1.

TABLE 1

| Parameters | Setting | |
|---|---|---|
| Apparatus LC | Shimadzu Nexera X2 LC-30 CE (Shimadzu Co., | |
| | Kyoto, Japan) | |
| Apparatus MS | QTrap 6500 (ABSciex, Framingham, | |
| | Massachussetts, USA) | |
| Column | BEH C18 column (2.1 mm × 100 mm, 1.7 μm | |
| | particle size) (Acquity UPLC, Waters) | |
| Column temperature | 40° | C. |
| Mobile phase (A) | 0.02% NH4OH in water | |
| Mobile phase (B) | Acetonitrile | |
| Flow rate | 0.4 | mL/min |
| Source | Electrospray (negative and positive mode) | |
| Ion spray voltage | −4500 V & 5500 V | |
| Ion source temperature | 650° | C. |

### LC-MS Method Validation

The whole method process was validated according to the guidelines provided by the USA Food and Drug Administration (FDA). For each validation day, 6 points calibration curves (ranges: 20-1000 pg/g for E1S) were prepared in duplicate with male cattle feces. Integrated peak area ratio between native hormones and marked internal standard was used to calculate response. Intraday precision and accuracy were evaluated by analyzing 5 levels of validation standards prepared in quintuplicate in cattle feces (standard concentration are gathered in Table 2). Interday precision and accuracy were determined by repeating the analysis two more times. Precision corresponds to the relative standard deviation (RSD=Standard deviation×100/Mean value) and accuracy to relative bias (RB=(Mean of observed concentrations−Spiked concentration)×100/Spiked concentration). Lower and upper limits of quantification were defined as the lowest and the highest concentrations, respectively, in the validation standards that reported RSD and RB lower than 15%. Extraction efficiencies and matrix effects were assessed on 2 levels (low: 50 pg/g for E1S and high: 500 pg/g for E1S), each level was assessed in triplicate. Extraction efficiency indicated the percentage of hormone extracted from the matrix and was computed by the ratio of the area measured after extraction and the area determined in post extraction spiked samples. Matrix effect reflects the effect of the matrix on the compound ionization, this effect could be positive (ionization enhancement) or negative (ionization suppression). Matrix effects were determined by the difference in percent between the ratio of area measured for the compounds and the area determined for the corresponding internal standard in a post extraction spiked standard and the same ratio for pure standards.

TABLE 2

| | Target | | |
|---|---|---|---|
| | (pg/g) | RSD | RB |
| Intra-day | | | |
| Level 1 | 20 | 12.0% | 2.9% |
| Level 2 | 30 | 7.8% | −1.9% |
| Level 3 | 50 | 9.0% | 2.3% |
| Level 4 | 800 | 5.4% | 0.0% |
| Level 5 | 1000 | 7.2% | 3.6% |
| Inter-day | | | |
| Level 1 | 20 | 10.9% | 8.6% |
| Level 2 | 30 | 5.5% | 6.1% |
| Level 3 | 50 | 11.8% | 5.2% |
| Level 4 | 800 | 8.0% | 1.6% |
| Level 5 | 1000 | 5.8% | 0.7% |

During the validation process, correlation coefficients (r²) for the calibration curve were observed to be greater than 0.99. Calibration curves were calculated using 1/x-weighted quadratic regression. The intra-and inter-day accuracy and precision determined during the validation process are summarized in Table 2. For each concentration tested, RSDs were less than 15% (RSD ranged from 5.4% to 12.0%), while RBs were less than 10% (ranging from −1.9% to 8.6%). Therefore, the lower limits of quantification (LOQ) of the method were set at 20 pg/g for E1S. Extraction efficiencies were 63% (CV: 22%) and 67% (CV: 11.2%) for E1S in low and high levels samples respectively. Matrix effects were 2.4% (CV: 13.7%) and −11.5% (CV: 5.2%) for E1S in low and high levels samples respectively.

### Statistics

Software R Studio was used (version 3.4.1; R Project for Statistical Computing) and statistical significance was established at p<0.05 for this double-blind prospective study. Normal distribution of values was tested with Shapiro-Wilk test. The spearman correlation test was used to determine correlations between serum and fecal hormones' concentrations. Correlation was only tested on the pregnant female cohort since E1S concentrations in feces were below the limit of quantification in all males and non-pregnant females.

### 2. Homogeneity of Fecal Matrix and Stability During Storage at Room Temperature

To assess homogeneity of fecal samples, 4 entire and unmixed feces from 4 different pregnant American Bison cows were collected shortly after the emission on the field and steroid hormones were determined in split samples taken from top, bottom, left and right part of each fecal sample. In order to assess stability of steroids in feces, in samples 1 and 2, the left part of the feces was homogenized and an aliquot was left at ambient temperature (15-25° C.) during 24 hours and another for 96 hours before being analyzed. Stability at ambient temperature was also assessed in feces from a non-pregnant female and another fecal sample from an American Bison bull.

Homogeneity of the feces was tested by measuring the steroid E1S in different parts of samples collected from pregnant females. The results are gathered in Table 3. The variation observed for E1S was moderate: coefficients of variation (CVs) ranging from 9.4% to 21.3%.

Regarding stability, in pregnant females, the steroid E1S levels increased over time. In non-pregnant females and males, E1S levels remained unquantifiable after 24 and 96 hours of incubation at room temperature (Table 4).

TABLE 3

| | Bison A | Bison B | Bison C | Bison D |
|---|---|---|---|---|
| | E1S (pg/g) | E1S (pg/g) | E1S (pg/g) | E1S (pg/g) |
| Top | 40.1 | 61.8 | 117.1 | 293.7 |
| Left | 52.5 | 50.6 | 154.5 | 252.1 |
| Right | 44.4 | 55.4 | 189.3 | 287.0 |
| Bottom | 43.2 | 51.3 | 131.3 | 324.3 |
| CV | 11.7% | 9.4% | 21.3% | 10.2% |

TABLE 4

| | Bison A | Bison B | Bison E | Bison F |
|---|---|---|---|---|
| | (pregnant) | (pregnant) | (not pregnant) | (male) |
| | E1S (pg/g) | E1S (pg/g) | E1S (pg/g) | E1S (pg/g) |
| 0 h | 40.1 | 61.8 | <20 | <20 |
| 24 h | 132.9 | 74.9 | <20 | <20 |
| 96 h | >1000 | >1000 | <20 | <20 |

### 3. Estrone-Sulfate (E1S) Measured in Feces

Table 5 gathers concentrations of estrone-sulfate (E1S) measured in feces of males, non-pregnant females and pregnant females. As it can be seen, E1S is a good biomarker to discriminate pregnant females from non-pregnant females and males. Indeed, E1S was not detected in any samples from males or non-pregnant females. In pregnant females, the median concentration was 250 pg/g, this concentration being superior to the lower limit of quantification (LOQ) set at 20 pg/g (see under the point “LC-MS method validation”) and so indicating the pregnancy of the female. In the context of the present invention, the lower limit of quantification (LOQ) corresponds to the predetermined threshold value estrone-sulfate steroid (E1S) concentration above which the ungulate is determined as being pregnant.

TABLE 5

| | E1S (pg/g) |
|---|---|
| | Median |
| Males (n = 4) | <LOQ |
| Pregnant females (n = 35) | 250 |
| Not pregnant females (n = 34) | <LOQ |

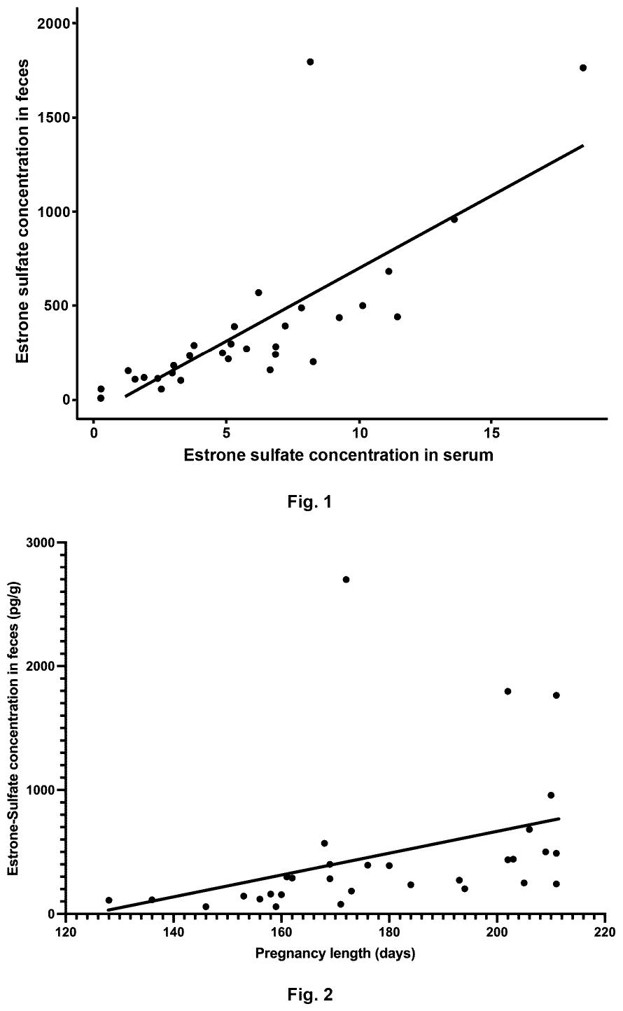

The correlation between serum and feces E1S concentration in pregnant females is significant (p<0.001) and good (r=0.77) (FIG. 1), confirming that E1S from feces is a good biomarker to discriminate pregnant females from non-pregnant females and males.

### 4. Evolution of Estrone-Sulfate (E1S) Concentration During Pregnancy

FIG. 2 illustrates the evolution of estrone-sulfate (E1S) concentrations measured in feces of pregnant females over time according to the LC/MS method described above. As it can be seen, the concentration of E1S in feces was correlated to the day of pregnancy (p<0.0001 and r=0.6748), meaning that a process according to the present invention allows the determination of the pregnancy stage of an ungulate from its feces.

The present invention has been described in terms of specific embodiments, which are illustrative of the invention and not to be construed as limiting. More generally, it will be appreciated by persons skilled in the art that the present invention is not limited by what has been particularly shown and/or described hereinabove.

Use of the verbs “to comprise”, “to include”, “to be composed of”, or any other variant, as well as their respective conjugations, does not exclude the presence of elements other than those stated.

Use of the article “a”, “an” or “the” preceding an element does not exclude the presence of a plurality of such elements.

1. A process for determining pregnancy and/or a pregnancy stage of an ungulate from its feces, comprising:
an extraction of steroids from an ungulate's feces sample previously taken or collected to obtain an extract comprising steroids,
a dosage of estrone-sulfate steroid (E1S) present in the extract by a separative method coupled to mass spectrometry to determine an estrone-sulfate steroid (E1S) concentration in the extract,
a determination of the pregnancy and/or of the pregnancy stage of the ungulate based on the estrone-sulfate steroid (E1S) concentration with reference to a predetermined threshold value estrone-sulfate steroid (E1S) concentration above which the ungulate is determined as being pregnant.

2. A process according to claim 1 wherein the ungulate's feces sample is stored at a temperature inferior to 15° C., preferably at a temperature inferior to 10° C., within 6 hours of taking or collecting the sample before the extraction of steroids from the feces sample.

3. A process according to claim 1 wherein the ungulate's feces sample is frozen at a temperature inferior to 0° C., preferably at a temperature inferior to −10° C., more preferably at a temperature ranging from −20° C. to −80° C., even more preferably at a temperature of −80° C., within 6 hours of taking or collecting the sample before the extraction of steroids from the feces sample.

4. A process according to claim 1 wherein the extraction of steroids from the ungulate's feces sample is performed with an organic solvent, for example with methanol.

5. A process according to claim 1 wherein the extract comprising steroids is washed with a washing solution, for example with hexane.

6. A process according to claim 1 wherein the separative method is a chromatographic method, for example gas chromatography or liquid chromatography or high-performance liquid chromatography or thin layer chromatography.

7. A process according to claim 1 wherein the predetermined threshold value estrone-sulfate steroid (E1S) concentration ranges from 10 pg/g to 500 pg/g.

8. A process according to claim 1 wherein testosterone as an additional steroid is dosed simultaneously to the dosage of estrone-sulfate steroid (E1S) in the extract by the separative method coupled to mass spectrometry to determine the concentration in the extract of said testosterone as an additional steroid.

9. A process according to claim 1 wherein the ungulate is the American Bison (Bison bison bison or Bison bison athabascae).

10. A kit for determining pregnancy and/or a pregnancy stage of an ungulate from its feces, comprising:
an extraction buffer for extracting steroids from an ungulate's feces sample,
a reconstitution buffer for reconstituting the extracted steroids in a precise volume of solution compatible with a separative method coupled to mass spectrometry,
a separation means, in particular a chromatographic column, for separating the steroids with the separative method coupled to mass spectrometry,
at least one mobile phase for transporting the extracted steroids through the separation means, in particular through the chromatographic column,
an internal standard mix being a mixture of isotopically labelled compounds, chemically similar to the steroids of interest, said internal standard mix comprising at least estrone-sulfate steroid (E1S) isotopically labelled,
a calibrator solution for calibrating the separative method coupled to mass spectrometry, said calibrator solution comprising estrone-sulfate steroid (E1S).

11. A kit according to claim 10, further comprising a precolumn.

12. A kit according to claim 10, further comprising at least one control solution for monitoring the accuracy and precision of the separative method coupled to mass spectrometry.

13. A kit according to claim 10, wherein the internal standard mix further comprises testosterone isotopically labelled.

14. A kit according to claim 10, further comprising a rinsing solution for rinsing the separation means and the system used for the separative method coupled to mass spectrometry.
